(19) United States
(12) Patent Application Publication
(10) Pub. No.: US 2019/0117532 A1
(43) Pub. Date: Apr. 25, 2019

(54) ORAL CARE COMPOSITIONS FOR PROMOTING GUM HEALTH

(71) Applicant: The Procter & Gamble Company, Cincinnati, OH (US)

(72) Inventors: Ross STRAND, Singapore (SG); Xiujun XU, Beijing (CN); Yang SU, Beijing (CN); Yunming SHI, Beijing (CN)

(21) Appl. No.: 15/788,255

(22) Filed: Oct. 19, 2017

(51) Int. Cl. A61K 8/19; A61Q 11/00; A61K 8/41; A61K 8/21

(52) U.S. Cl. CPC A61K 8/19; A61Q 11/00; A61K 8/41; A61K 2800/92; A61K 2800/48; A61K 2800/30; A61K 8/21

(57) ABSTRACT

Oral care compositions comprising an antifibrinolytic agent (e.g., tranexamic acid) and stannous ion source are provided for promoting Gum Health of a consumer.

### FIELD OF THE INVENTION

The present invention relates to oral care compositions comprising stannous ion source and an antifibrinolytic agent for promoting Gum Health of a consumer. In particular, such oral care compositions are useful for improving gingival wound healing and improving the reduction of bacterial activity in the oral cavity of the consumer.

### BACKGROUND OF THE INVENTION

Gum disease, such as gingivitis and/or periodontitis, gives rise to acute and chronic gum inflammation in the oral cavity. “Gingivitis” is the milder form of the disease. Symptoms of gingivitis may include: gingival bleeding; and redness, swollen, or tender gums. If left untreated, gingivitis can advance to “periodontitis”. With periodontitis, gums pull away from the teeth and form spaces called “periodontal pockets” that can become infected by pathogenic bacteria. The bacteria are present on the tooth root surfaces as biofilms. The bacteria in the biofilms can attack the gingival and underlying alveolar bone supporting teeth. These attacks can cause major damage to the soft tissue and bone that support teeth. In the later stage of gum disease (i.e., “advanced periodontitis”), more serious problems of loosening of teeth and eventual tooth loss can occur.

Some commercially available oral care compositions aim, principally, at alleviating one or more symptoms of the earlier stage of gum disease (i.e., gingivitis), which includes: relief of red, swollen, or tender gums; and/or stem gum bleeding. Typically, these compositions claim benefits such as, “gum care”, “oral care”, “oral health”, “dental care,” or “dental health” to consumers. An example of such a composition is “Colgate® Total” toothpaste, which they claim to “help reduce the first stage of gum disease”, which is defined as “gingivitis, or bleeding gums” (see http://www.colgatetotal.com/total-benefits/whole-mouth-health/gingivitis-control). To help distinguish the benefits of the commercially available oral care compositions versus the present invention, the inventors herein refer to the aforementioned benefits of these commercially available oral care compositions collectively as “Gum Care”. This is because these commercially available oral care compositions have been formulated primarily to care for the gums and relieve the symptoms (e.g., gum bleeding; and/or redness, swelling, or tender gums) associated with the earlier stage of gum disease (i.e., gingivitis).

However, there is a need to provide overall “Gum Health” benefits, which as used herein, is a broader term and is intended to encompass at least some of the aforementioned Gum Care benefits, as well as providing additional anti-bacterial benefits to mitigate the harmful effects of bacteria as it relates to gum disease, including gingivitis, periodontitis, or both.

There is at least one of several drawbacks to the above described conventional approaches. Firstly, these commercially available oral care compositions may promote Gum Care, but they do not go far enough to also promote Gum Health. In fact, these commercially available oral care compositions generally fail to provide any significant anti-bacterial effects in addition to the Gum Care benefits (e.g., anti-bleeding and/or anti-swelling). This is a problem because if the bacteria in the biofilms are not controlled, they can then increase the size of the periodontal pockets leading to periodontitis. Secondly, Gum Health may correlate to overall body health. In other words, an individual's Gum Health can be an indicator of the person's overall body health. Studies suggest that the risk of developing any one (or more) of these potential life threatening conditions such as, for example, heart disease and stroke, diabetes, kidney disease, preterm birth, and/or osteoporosis, may increase as overall Gum Health decreases (see U.S. Pat. No. 6,846,478; Doyle, M. J.; & U.S. Pat. No. 8,283,135; Doyle, M. J.). Thus, it is desirable to improve overall Gum Health, not just Gum Care, in order to ensure better overall body health.

Other commercially available oral care compositions may contain an anti-bacterial agent aimed at controlling the growth of bacteria in the biofilms. Some oral compositions may also contain tranexamic acid and a fluoride source. Tranexamic acid has been described as an anti-bleeding agent useful for gingival wound healing. It has also been described to inhibit gingival inflammation and/or acts as an anti-swelling agent (see U.S. Pat. No. 4,272,513; Gaffar, A.; & U.S. Pat. No. 4,272,512; Gaffar, A.). Fluoride has been well-documented to prevent and control dental caries. One disadvantage of tranexamic acid-containing oral care compositions is that the tranexamic acid can become unstable and loses its activity upon aging. Furthermore, the unstable tranexamic acid can discolor the oral care compositions, turning them yellow to dark brown, which is undesirable to the consumers. Previous oral care compositions that have described the instability and discoloration issues have formulated with tranexamic acid at higher levels of from 1.5% to 2% (see examples in U.S. Pat. No. 4,272,513; Gaffar, A., & U.S. Pat. No. 4,272,512; Gaffar, A.). According to those references, the higher levels are required in order to provide sufficient tranexamic acid efficacy for the wound healing and other benefits. To overcome the problems, these previous attempts have resorted to adding additional components such as, for example, TiO₂, folic acid or flavor mixtures (e.g., methyl salicylate, menthol, eugenol and cineol) to prevent the above-described discoloration in storage without significantly diminishing the stability and activity of the tranexamic acid. However, there is a continuing need to simplify formulations and processing steps to provide cost effective and efficacious toothpaste and other oral care formulations.

Anti-bacterial agents useful in these other commercially available oral care compositions may include such as, for example: zinc citrate (e.g., Darlie™ Expert toothpaste), IPMP (e.g., Lion™ Systema toothpaste), and cetylpyridinium chloride (CPC). Unfortunately, these anti-bacterial agents may have poor penetration into the biofilms. As a result, these anti-bacterial agents may simply chemically degrade before being able to interact with bacteria deep in the biofilms. Alternatively, the anti-bacterial agents may exhibit a markedly slow penetration rate into the biofilms. As a result, these anti-bacterial agents may have an inefficient rate of penetration in the biofilms and will take longer to engage and kill the bacteria. It is believed that one or more of these drawbacks render these oral care compositions less efficacious to promote Gum Health, particularly as it relates to reducing bacteria activity.

One solution is to add higher amounts of the anti-bacterial agents to accommodate for their degradation and/or sub-optimal or inefficient penetration rate in the biofilms. However, the amounts required of these anti-bacterial agents in the oral care products to provide efficacy may be unreasonably high given their relatively high Minimum Inhibitory Concentrations (“MIC”) (data not shown). The MIC is the minimum concentration in micrograms per milliliter of an anti-bacterial agent at which no bacterial growth is observed. At concentrations below the MIC, an anti-bacterial agent is ineffective at killing or inhibiting the growth and reproduction of bacteria. At concentrations above the MIC, an anti-bacterial agent is effective at killing or inhibiting the growth and reproduction of bacteria.

This in turn may be disadvantageous for several aspects, including limitations to the oral care products efficacy due to maximum upper safety limits or cost constrains of using ever increasing amounts of these anti-bacterial agents, and/or adverse side effects that may be associated from higher levels of these anti-bacterial agents. In short, due to the chemical degradation and/or sub-optimal or inefficient penetration rate of these anti-bacterial agents into the biofilms, there is a continuing need for an improved oral care composition that promotes Gum Health benefits. It is desirable if this new oral care composition has improved anti-bacterial action in biofilms.

Accordingly, there is a need to provide an oral care composition that provides Gum Health benefits to consumers, or at least provide better associated Gum Health benefits (e.g., gingival wound healing and anti-bacterial benefits) than those compositions that are commercially available.

### SUMMARY OF THE INVENTION

The present invention attempts to address this need based, at least in part, on the surprising discovery that the combination of an antifibrinolytic agent, such as tranexamic acid, and a stannous ion source in an oral care composition promotes Gum Health benefits that include at least gingival wound healing and anti-bacterial benefits. In particular, the oral care composition comprises tranexamic acid for gingival wound healing, and stannous ion source as an anti-bacterial agent to combat the undesirable effects of bacteria activity in the oral cavity.

One advantage of the present invention is “better deep biofilm penetration and/or bacteria kill”. To this end, it is further surprisingly found that the penetration depth and/or penetration rate of stannous ion into the biofilms may be increased, when used in combination with tranexamic acid. In short, the synergistic combination of tranexamic acid and stannous ion source in the oral care composition may be such that an improvement in the Gum Health benefit is achieved. Furthermore, the use of the oral care compositions of the present invention may provide the consumers an improved Gum Health benefit.

Another advantage of the present invention is to provide oral care compositions for promoting Gum Health as it relates to the totality of symptoms associated with gingivitis, periodontitis, or both. It is yet a further advantage that the oral care compositions of the present invention have improved Gum Health benefits. It is yet a further advantage of the present invention to provide oral care compositions having improved penetration depth of the anti-bacterial agent(s) into the biofilms. It is yet a further advantage of the present invention to provide oral care compositions having improved penetration rate of the anti-bacterial agent(s) into the biofilms. It is yet a further advantage of the present invention to provide cost effective and efficacious oral care compositions for promoting Gum Health. It is yet a further advantage that the oral care composition, is a dentifrice, and preferably provides pleasant taste and mouth-feel experience. It is yet a further advantage that the oral care compositions have physical and chemical stability across a range of manufacturing, handling and storage conditions. It is yet a further advantage that the oral care compositions have a stable quality of end product (e.g., consistent visual appearance and no discoloration, gingival wound healing performance, etc.) even after three months storage at 40° C. It is yet a still further advantage that the oral care compositions of the present invention minimize the use of antibacterial agents. It is yet a still further advantage that the oral compositions of the present invention minimize the amount of the antifibrinolytic agent to reduce and/or eliminate the instability and/or discoloration problems as described above.

In one aspect, the present invention is directed to an oral care composition comprising: a) from 0.01% to 5%, preferably from 0.05% to 4%, by weight of the composition, of a stannous ion source; and b) from 0.01% to 10%, preferably less than 5%, more preferably less than 3%, still more preferably less than 1%, by weight of the composition, of an antifibrinolytic agent.

In another aspect, the present invention is directed to an oral care composition comprising: a) from 0.01% to 5%, preferably from 0.05% to 4%, by weight of the composition, of a stannous ion source; and b) from 0.01% to 10%, preferably less than 5%, more preferably less than 3%, still more preferably less than 1%, by weight of the composition, of one or more compounds selected from the group consisting of tranexamic acid, epsilon aminocaproic acid, p-aminomethylbenzoic acid, and combinations thereof.

In another aspect of the present invention, the above mentioned oral care composition further comprising c) a thickening agent, preferably from 0.01% to 5%, preferably from 1% to 2.5%, by weight of the composition, of a thickening agent comprising at least one agent, preferably at least two agents, selected from the group consisting of: (i) a linear sulfated polysaccharide; (ii) a natural gum; (iii) a non-ionic cellulose derivative; (iv) a polyvinyl pyrrolidone (PVP); (v) polymers comprising at least a a polycarboxylated ethylene backbone; (vi) polyacrylamide; (vii) co-polymers with acrylamide; (viii) pectin; (ix) proteins; (x) polyethylene glycols (PEG), preferably high molecular weight PEG; and (xi) combinations thereof.

In yet another aspect of the present invention, the above mentioned oral care composition further comprising d) from 0% to less than 0.001%, by weight of the composition, or preferably is free or substantially free, of folic acid.

In yet still another aspect of the present invention, a method is provided for promoting Gum Health in a human subject comprising administering to the subject's oral cavity an oral care composition of the present invention, preferably once a day, more preferably twice a day.

These and other features of the present invention will become apparent to one skilled in the art upon review of the following detailed description when taken in conjunction with the appended claims.

### BRIEF DESCRIPTION OF THE DRAWINGS

While the specification concludes with claims that particularly point out and distinctly claim the invention, it is believed the present invention will be better understood from the following description of the accompanying figures.

FIG. 1 is a timeline for assessing Mazza indexed bleeding sites for the Assay for Measuring Improve Gingival Wound Healing.

FIG. 2 are bar graphs providing Mazza indexed number of bleeding sites at various time intervals (Baseline, Day 3 and Week 4) for Inventive Composition Ex. 2 (comprising stannous and an antifibrinolytic), and a positive and negative control.

FIG. 3 is a perspective view of an oral splint with hydroxyapatite (“HA”) disks attached thereto.

FIG. 4 is a perspective view of the HA disk having grooves therein.

FIG. 5 is a schematic of a cross sectional view of the groove with biofilm therein.

FIG. 6 are images of the wound healing of Human Gingival Fibroblasts at 24 hrs post-treatment with Inventive Composition Ex. 2 (containing stannous and TA) or Control Composition Ex. 9 (Sn only).

### DETAILED DESCRIPTION OF THE INVENTION

### Definitions

As used herein, the articles including “a” and “an” when used in a claim, are understood to mean one or more of what is claimed or described.

The terms “alleviate” and “alleviating” are used interchangeably and means minimizing, preventing, delaying, and/or treating at least one symptom of gum disease to effect positive change (i.e., benefit) to the consumer.

The term “biofilms” as used herein means a matrix-enclosed bacterial population adherent to each other and/or to surfaces or interfaces in the oral cavity.

The term “comprising” as used herein means that steps and ingredients other than those specifically mentioned can be added. This term encompasses the terms “consisting or and “consisting essentially of”.” The compositions of the present invention can comprise, consist of, and consist essentially of the essential elements and limitations of the invention described herein, as well as any of the additional or optional ingredients, components, steps, or limitations described herein.

The term “dentifrice” as used herein means paste, gel, powder, tablets, or liquid formulations, unless otherwise specified, that are used to clean the surfaces of the oral cavity.

The term “free” as used herein refers to no amount of that material is present in the composition.

The term “Gum Care” as used herein refers to inherent or promoted benefits of an oral care composition directed, principally, to alleviating one or more symptoms associated with an early stage of gum disease (i.e., gingivitis). Such symptoms may include, for example bleeding gums; and red, swollen, or tender gums.

The term “Gum Health” as used herein refers to inherent or promoted benefits of an oral care composition to provide “Gum Care” benefits that include at least improve gingival wound healing, as well as, providing additional improve reduction of bacterial activity to mitigate the harmful effects of bacteria as it relates to gum disease, including gingivitis, periodontitis or both.

The term “improve gingival wound healing” as used herein means reduce gum bleeding in the oral cavity as determined by any generally accepted in vitro or in vivo gum bleeding assay or test or the assay described in Example 11.

The term “improve reduction of bacterial activity” as used herein means reduce bacterial activity in the oral cavity as determined by any generally accepted in vitro or in vivo anti-bacterial assay or test; or preferably the assay described in Example 12 and the in-vitro wound healing assay described in Example 13.

The term “oral care composition” or “oral care compositions” as used herein means a product that in the ordinary course of usage is retained in the oral cavity for a time sufficient to contact some or all of the dental surfaces and/or oral tissues for purposes of oral activity. In one embodiment, the composition provides a benefit when used in the oral cavity. The oral care composition of the present invention may be in various forms including toothpaste, dentifrice, tooth gel, tooth powders, tablets, rinse, mouthwash, sub gingival gel, foam, mouse, chewing gum, lipstick, sponge, floss, prophy paste, petrolatum gel, denture adhesive, or denture product. In one embodiment, the oral composition is in the form of a paste or gel. In another embodiment, the oral composition is in the form of a dentifrice. The oral composition may also be incorporated onto strips or films for direct application or attachment to oral surfaces, or incorporated into floss.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The term “promoting” as used herein means to promote and/or enhance the Gum Health benefits associated with using the oral care compositions of the present invention in the oral cavity.

The term “substantially free” as used herein refers to no intentional amount of that material is added to the composition or an amount of a material that is less than 0.05%, 0.01%, or 0.001% of the composition.

The term “synergistic Gum Health benefit” as used herein means analytically measurable increases in any two Gum Health benefits that include at least improve gingival wound healing and improve reduction of bacterial activity in the oral cavity, that is more than additive.

The term “teeth” as used herein refers to natural teeth as well as artificial teeth or dental prosthesis.

The term “total water content” as used herein means both free water and water that is bound by other ingredients in the oral care composition.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified.

All measurements referred to herein are made at 25° C. (i.e., room temperature) unless otherwise specified.

### Oral Care Compositions

It has been surprisingly discovered that the combination of an antifibrinolytic agent, preferably tranexamic acid, and stannous ion (i.e., an anti-bacterial agent) in an oral care composition is particularly useful for promoting Gum Health benefits to consumers. In particular, the surprising discovery was that the penetration of the stannous ion into the biofilms is markedly improved when combined with the tranexamic acid. Without wishing to be bound by theory, the tranexamic acid contains both carboxylic and amine groups. It is believed that the stannous ions can bind strongly to these chemical moieties on the tranexamic acid to positively influence the penetration of stannous ions into the biofilms.

It has also been surprisingly found that the penetration depth and/or the penetration rate of stannous ions into the biofilms may be increased, or markedly increased, when formulated with tranexamic acid. In short, the presence of tranexamic acid in combination with stannous ion source in an oral care composition aids the composition's efficacy in mediating the harmful effects of the bacteria in the biofilms on the gums.

In one aspect, the present invention is directed to an oral care composition comprising: a) from 0.01% to 5%, preferably from 0.05% to 4%, by weight of the composition, of a stannous ion source; b) from 0.01% to 10%, preferably less than 1%, by weight of the composition, of an antifibrinolytic agent. Preferably, the oral care composition further comprises from 0.01% to 5%, preferably from 1% to 2.5%, by weight of the composition, of a thickening agent comprising at least one agent, preferably at least two agents, selected from the group consisting of: (i) a linear sulfated polysaccharide; (ii) a natural gum; (iii) a non-ionic cellulose or derivative thereof; (iv) a polyvinyl pyrrolidone (“PVP”); (v) a polymer comprising at least a polycarboxylated ethylene backbone; (vi) polyacrylamide; (vii) co-polymers with acrylamide; (viii) pectin; (ix) proteins; (x) polyethylene glycols (“PEG”), preferably high molecular weight PEG; and (xi) combinations thereof.

### Anti-Bacterial Agent

The present invention relates to the above mentioned oral care compositions comprising, in a preferred embodiment, the stannous ion source present in the amount of from 0.01% to 5%, preferably from 0.05% to 4%, or more preferably from 0.1% to 2%, by weight of the composition, to provide anti-bacterial effectiveness. The stannous ion source used herein may include any safe and effective stannous salt. Suitable examples of stannous ion source is selected from the group consisting of stannous chloride, stannous fluoride, stannous acetate, stannous gluconate, stannous oxalate, stannous sulfate, stannous lactate, stannous tartrate, stannous iodide, stannous chlorofluoride, stannous hexafluorozirconate, stannous citrate, stannous malate, stannous glycinate, stannous carbonate, stannous phosphate, stannous pyrophosphate, stannous metaphosphate, and combinations thereof. Preferably, the stannous ion source is selected from stannous fluoride, stannous chloride, and combinations thereof. More preferably the stannous ion source comprises stannous chloride. Other examples of stannous salts are found in U.S. Pat. No. 5,578,293; Prencipe, M., & U.S. Pat. No. 5,281,410; Lukacovic, M. F. In addition to the stannous ion source, other ingredients used to stabilize the stannous ions may be included, such as the ingredients described in U.S. Pat. No. 5,004,597; Majeti, S., & U.S. Pat. No. 5,578,293; Prencipe, M.

The oral care compositions of the present invention may optionally also include other anti-bacterial agents present in an amount of from 0.035% or more, from 0.05% to 2%, from 0.1% to 1%, by weight of the composition. Examples of these other anti-bacterial agents may include non-cationic anti-bacterial agents such as, for example, halogenated diphenyl ethers, phenolic compounds including phenol and its homologs, mono and poly-alkyl and aromatic halophenols, resorcinol and its derivatives, xylitol, bisphenolic compounds and halogenated salicylanilides, benzoic esters, and halogenated carbanilidies. Also useful anti-bacterial agents are enzymes, including endoglycosidase, papain, dextranase, mutanase, and combinations thereof. Such agents are disclosed in U.S. Pat. No. 2,946,725; Norris, P. E., & U.S. Pat. No. 4,051,234; Gieske, H. A. Examples of other anti-bacterial agents include chlorhexidine, and flavor oils such as thymol. In another example, the other anti-bacterial agent can include triclosan (5-chloro-2-(2,4-dichlorophenoxy)phenol).

### Antifibrinolytic Agent

The present invention further relates to the above mentioned oral care compositions comprising, in a preferred embodiment, an antifibrinolytic agent. Preferably, the antifibrinolytic agent is a compound of formula (I):

H₂N—X—COOH (I)

wherein X is a branched or unbranched, saturated or unsaturated, aliphatic group; or an aromatic group.

The term “aliphatic group” as used herein, includes both saturated and unsaturated, unbranched (i.e., straight chain) and branched, acyclic, cyclic, or polycyclic aliphatic hydrocarbons containing 1 to 20 carbon atoms, which are optionally substituted. Aliphatic group is intended herein to include, but is not limited to groups such as alkyl, alkenyl, alkynyl, alkylene, alkenylene, alkynylene and cycloalkyl moieties.

The term “aromatic group” as used herein refers to an aryl ring and includes, but are not limited to, monocyclic or multi-cyclic hydrocarbon ring system consisting only of hydrogen and carbon and containing from 6 to 18 carbon atoms, where the ring system may be partially saturated.

Unless stated otherwise in the specification, the aryl ring is optionally substituted. Aromatic group is intended herein to include, but is not limited to groups such as phenyl, naphthyl, and fluorenyl.

The term “cyano” refers to the —CN functional group.

The terms “halo” or halogen by themselves or as part of another substituent refers to fluorine, chlorine, bromine, or iodine atom.

The term “oxo” refers to the ═O substituent.

“Alkyl” refers to a group containing a straight or branched hydrocarbon chain consisting solely of carbon and hydrogen atoms, containing no unsaturation, having from 1 to 20 carbon atoms, preferably 1 to 12 carbon atoms, preferably 1 to 8, or preferably 1 to 6 carbon atoms, and which is attached to the rest of the molecule by a single bond, e.g., methyl, ethyl, propyl, 1-methylethyl (iso-propyl), butyl, pentyl, and the like. An alkyl may be optionally substituted.

“Alkenyl” refers to a group containing straight or branched hydrocarbon chain consisting solely of carbon and hydrogen atoms, containing at least one carbon-carbon double bond, having from 2 to 20 carbon atoms, preferably 2 to 12 carbon atoms, or preferably 1 to 8 carbon atoms, e.g., ethenyl, prop-2-enyl, but-1-enyl, pent-1-enyl, penta-1,4-dienyl, and the like. An alkenyl may be optionally substituted.

“Alkynyl” refers to a group containing straight or branched hydrocarbon chain consisting solely of carbon and hydrogen atoms, containing at least one carbon-carbon triple bond, having from 2 to 20 carbon atoms, preferably 2 to 12 carbon atoms, or preferably 1 to 8 carbon atoms, e.g., ethynyl, propynyl, butynyl, pentynyl, hexynyl, and the like. An alkynyl may be optionally substituted.

“Alkylene” or “alkylene chain” refers to a group containing straight or branched hydrocarbon chain linking the rest of the molecule to a group, consisting solely of carbon and hydrogen, containing no unsaturation and having from 1 to 12 carbon atoms, e.g., methylene, ethylene, propylene, butylene, and the like. An alkylene may be optionally substituted.

“Alkenylene” or alkenylene chain” refers to a straight or branched hydrocarbon chain linking the rest of the molecule to a group, consisting solely of carbon and hydrogen, containing at least one carbon-carbon double bond and having from 2 to 20 carbon atoms, preferably 2 to 12 carbon atoms, e.g., ethenylene, propenylene, butenylene, and the like. An alkenylene may be optionally substituted.

“Alkynylene” or “alkynylene chain” refers to a straight or branched hydrocarbon chain linking the rest of the molecule to a group, consisting solely of carbon and hydrogen, containing at least one carbon-carbon triple bond and having from 2 to 20 carbon atoms, e.g., propynylene, butynylene, and the like. An alkynylene may be optionally substituted.

“Cycloalkyl” refers to a stable saturated mono-cyclic or polycyclic hydrocarbon group consisting solely of carbon and hydrogen atoms, which may include fused or bridged ring systems, having from 3 to 15 carbon atoms, preferably having from 3 to 10 carbon atoms or preferably from 3 to 7 carbon atoms, e.g., cyclohexane. A cycloalkyl may be optionally substituted.

“Haloalkyl” refers to an alkyl as defined above that is substituted by one or more halogen groups, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. A haloalkyl may be optionally substituted.

“Heterocyclyl” refers to a stable 3- to 24-membered saturated ring which consists of 2 to 20 carbon atoms and from 1 to 6 heteroatoms selected from atoms consisting of nitrogen, oxygen, or sulfur. Unless stated otherwise specifically in the specification, the heterocyclyl may be a monocyclic, bicyclic, tricyclic or tetracyclic ring system, which may include fused or bridged ring systems; and the nitrogen, carbon or sulfur atoms in the heterocyclyl may be optionally oxidised; the nitrogen atom may be optionally quaternised. A heterocyclyl may be optionally substituted.

“Heterocyclylalkyl” refers to a functional group of the formula —RₐRₑ where Rₐ is an alkylene as defined above and Rₑ is a heterocyclyl as defined above, and if the heterocyclyl is a nitrogen-containing heterocyclyl, the heterocyclyl may be attached to the alkylene at the nitrogen atom. A heterocyclylalkyl may be optionally substituted.

“Heteroaryl” refers to a 5- to 20-membered aromatic ring which consists of 1 to 17 carbon atoms and from 1 to 3 heteroatoms selected from atoms consisting of nitrogen, oxygen and sulfur. The heteroaryl may be a monocyclic, bicyclic, tricyclic or tetracyclic ring system, which may include fused or bridged ring systems. A heteroaryl may be optionally substituted.

“Heteroarylalkyl” refers to a functional group of the formula —RₐR_{f} where Rₐ is an alkylene as defined above and R_{f} is a heteroaryl as defined above. A heteroarylalkyl may be optionally substituted.

“Optionally substituted” means that the subsequently described event of circumstances may or may not occur and that the description includes instances where the event or circumstance occurs and instances in which it does not. For example, unless specified otherwise, “optionally substituted” means that the chemical moiety may or may not be substituted by one or more of the following groups: alkyl, alkenyl, halo, haloalkenyl, cyano, nitro, aryl, cycloalkyl, heterocyclyl, heteroaryl, oxo, —OC(O)—R¹, —N(R¹)₂, —C(O)R′, —C(O)OR′, —C(O)N(R¹)₂, —N(R¹)C(O)OR², —N(R¹)C(O)R², —N(R¹)S(O)ₜR² (where t is 1 to 2), —S(O)ₜOR² (where t is 1 to 2), —S(O)ₓR² (where x is 0 to 2) and —S(O)ₜN(R¹)₂ (where t is 1 to 2) where each R¹ is independently hydrogen, alkyl, haloalkyl, cycloalkyl, cycloalkylalkyl, aryl (optionally substituted with one or more halogen groups), aralkyl, heterocyclyl, heterocyclylalkyl, heteroaryl or heteroarylalkyl; and each R² is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted unless otherwise indicated.

Preferably, the antifibrinolytic agent is selected from tranexamic acid, epsilon aminocaproic acid, p-aminomethylbenzoic acid, or combinations thereof. Preferably, the antifibrinolytic agent is present in the amount of from 0.1% to less than 1%, preferably from 0.2% to 0.7%, or more preferably from 0.25% to 0.55%, by weight of the composition. Preferably, the antifibrinolytic agent is tranexamic acid (“TA”), which has the chemical name trans-4-aminomethyl-cyclohexanecarboxylic acid (CAS number 1197-18-8) and the following structural formula (II):

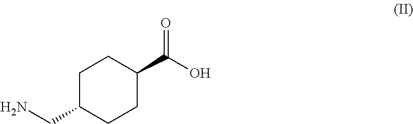 [SMILES: NC[C@H]1CC[C@H](C(=O)O)CC1]

Tranexamic acid is an antifibrinolytic agent used to prevent lysis or dissolution of fibrin clots. It assists in stabilizing fibrin clots, which maintains coagulation and assists in the control of bleeding. Tranexamic acid has been described as non-antibacterial and highly effective for inhibiting gingival inflammation, bleeding, and/or swelling (see U.S. Pat. No. 4,272,513; Gaffar, A.). The term “tranexamic acid” includes the acid itself or bioprecursors or salts thereof having the desired anti-bleeding activity. Preferably, tranexamic acid may be employed in free acid form or in the form of an orally acceptable salt thereof, preferably water soluble, such as with an alkali metal (e.g., Na or K), ammonium, or C₁-C₈ mono-, di-, or tri-substituted ammonium (e.g., alkanol substituted such as mono-, di-, or tri-ethanolammonium) cation. Tranexamic acid is commercially available as Cyclokapron®. Alternatively, the tranexamic acid may be synthesized or isolated from cis-trans mixtures thereof (see U.S. Pat. No. 3,499,925; Naito, T.).

It has been discovered that the above mentioned tranexamic acid stability and discoloration problems can be resolved by lowering the tranexamic acid concentration in the compositions. It is surprisingly found that only minimal reduction, such as less than 1%, by weight of the composition, of tranexamic acid can make material differences in its stability and formulation compatibility. The reduced levels of tranexamic acid does not negatively impact its activity and/or efficacy for gingival wound healing due to the effect, potentially synergistic effect, of combining it with stannous ions in preferred oral care compositions of the present invention. Without wishing to be bound by theory, it is believed that stannous ions can bind strongly to these chemical moieties on the tranexamic acid to positively influence the penetration to maintain sufficient efficacy at lower concentrations.

In another embodiment, the antifibrinolytic agent is epsilon aminocaproic acid (“EACA”). Preferably, the EACA is present in the amount of from 0.01% to 5%, preferably from 0.1% to 1%, or more preferably less than 1%, by weight of the composition. EACA has the chemical name hexanoic acid, 6-amino-(CAS number 60-32-2) and the following structural formula (III):

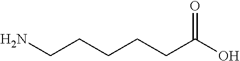

Epsilon aminocaproic acid and its analogue are commercially available under the tradename Amicar®. The term “epsilon aminocaproic acid” includes the acid itself or bioprecursors or salts thereof having the desired anti-bleeding activity. It is a derivative of the amino acid lysine and is an antiplasmin agent. Similar to tranexamic acid, the use of epsilon aminocaproic acid can also introduce a problem of discoloration into the oral care composition (see U.S. Pat. No. 4,649,044; Gomi, T.). Previous oral care compositions that have described the instability and discoloration issues have formulated with epsilon aminocaproic acid at levels of from 0.05% to 1% (see examples in U.S. Pat. No. 4,272,513; Gaffar, A., & U.S. Pat. No. 4,272,512; Gaffar, A.). Without wishing to be bound by theory, it is believe that the discoloration is attributable to reaction of the epsilon aminocaproic acid with an aldehyde group in the flavorant component. To avoid discoloration, the above reference proposes introducing cyclodextrin and use of a flavor component substantially free of an aldehyde group. However, that solution is not desirable as it complicates the formulation and limits the type of flavorant materials that can be used. Instead, the present invention advocates combining epsilon aminocaproic acid with a stannous ion to overcome the above mentioned problem.

In yet another embodiment, the antifibrinolytic agent is p-aminomethylbenzoic acid (“PAMBA”). Preferably, PAMBA is present in the amount of from 0.01% to 5%, preferably from 0.1% to 1%, or more preferably less than 1%, by weight of the composition. The p-aminomethylbenzoic acid (CAS number 56-91-7) has the following structural formula (IV):

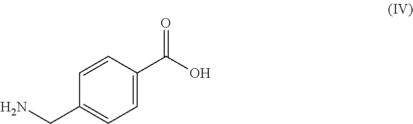 [SMILES: NCc1ccc(C(=O)O)cc1]

The term “p-aminomethylbenzoic acid” includes the acid itself or bioprecursors or salts thereof having the desired anti-bleeding activity.

### Thickening Agent

Preferably, the oral care compositions of the present invention herein may include one or more thickening agents, more preferably at least two thickening agents. Preferably, the thickening agents are present in the amount of from 0.01% to 10%, preferably from 1% to 2.5%, by weight of the composition. In particular, the present invention further relates to the above mentioned oral care compositions comprising, in a preferred embodiment, a thickening agent selected from the group consisting of:
(i) the liner sulfated polysaccharide is a carrageenan;
(ii) the natural gum is selected from the group consisting of xanthan gum, gum karaya, gum arabic, gum tragacanth, and combinations thereof;
(iii) the non-ionic cellulose or derivative thereof having an average molecular weight range of 50,000 to 1,300,000 Daltons, and preferably an average degree of polymerization from 300 to 4,800;
(iv) the polymer comprising at least a polycarboxylated ethylene backbone and selected from the group consisting of: co-polymers of maleic anhydride with methyl vinyl ether having a molecular weight of 30,000 to 1,000,000 Daltons; homo-polymers of acrylic acid; and co-polymers of maleic acid and acrylic acid or methacrylic; and
(v) combinations thereof.

Preferably, the oral care composition according to the present invention, wherein the thickener is selected from the group consisting of: (i) the carrageenan is selected from the group consisting of Kappa-carrageenan, Iota-carrageenan, Lambda-carrageenan, and combinations thereof; (ii) the natural gum is xanthan gum; (iii) the non-ionic cellulose or derivative thereof is hydroxyethyl cellulose (“HEC”); (iv) the co-polymers of maleic anhydride with methyl vinyl ether are at least one of: Gantrez AN139 (M.W. 500,000 daltons), Gantrez AN119 (M.W. 250,000 daltons), or S-97 Pharmaceutical Grade (M.W. 70,000 daltons); and the homo-polymers of acrylic acid and co-polymers of maleic acid and acrylic acid or methacrylic acid are at least one of: Acusol 445, Acusol 445N, Accusol 531, Acusol 463, Acusol 448, Acusol 460, Acusol 465, Acusol 490, Sokalan CPS, Sokalan CP7, Sokalan CP45, or Sokalan CP12S; and (v) combinations thereof.

In an example, the GANTREZ™ series of polymers are co-polymers of maleic anhydride with methyl vinyl ether having a molecular weight (M.W.) of 30,000 daltons to 1,000,000 daltons. These co-polymers are available for example as GANTREZ™ AN139 (M.W. 500,000 daltons), AN119 (M.W. 250,000 daltons) and S-97 Pharmaceutical Grade (M.W. 70,000 daltons), from Ashland Chemicals (Kentucky, USA).

In another example, the ACUSOL™ and the SOKALAN series of polymers include homopolymers of acrylic acid and copolymers of maleic acid and acrylic acid or methacrylic. Examples are 0:1000 to 1000:0 copolymers of maleic acid with acrylic acid having a molecular weight (M.W.) of about 2,000 to about 1,000,000. These copolymers are commercially available as ACUSOL™ 445 and 445N, ACUSOL™ 531, ACUSOL™ 463, ACUSOL™ 448, ACUSOL™ 460, ACUSOL™ 465, ACUSOL™ 497, ACUSOL™ 490 from Dow Chemicals (Michigan, USA) and as Sokalan® CP 5, Sokalan® CP 7, Sokalan® CP 45, and Sokalan® CP 12 S from BASF (New Jersey, USA).

### Anti-Caries Agent

The oral care compositions may include an effective amount of an anti-caries agent. In one aspect, the anti-caries agent is a fluoride ion source. The fluoride ion source may be present in an amount sufficient to provide fluoride ions concentration in the composition at 25° C., and/or can be used at levels of from 0.0025% to 2%, or preferably from 0.5% to 1.5%, by weight of the composition, to provide anti-caries effectiveness. A wide variety of fluoride ion-yielding materials can be employed as sources of soluble fluoride in the present invention. Examples of fluoride ions are disclosed in U.S. Pat. No. 3,535,421; Briner, W. W., & U.S. Pat. No. 3,678,154; Widder, J. S. Suitable examples of fluoride ions may be selected from a source comprising sodium fluoride, indium fluoride, amine fluoride, sodium monofluorophosphate (“MFP”), potassium fluoride, zinc fluoride, and mixtures thereof.

### Anti-Plaque Agent

The oral care composition may include one or more of an anti-plaque or anti-tartar agent present in the amount of from 0.001% to 20%, or from 0.1% to 5%, by weight of the composition. Non-limiting examples may include pyrophosphate salt as a source of pyrophosphate ion. Other examples are disclosed in U.S. Pat. No. 8,691,190; Haught, J. C., paragraph 55.

pH

The pH of the oral care composition may be from 4.5 to 11, or preferably from 5 to 10. Depending upon the actives used in the oral care composition, a different pH may be desired. For formulations containing Stannous Fluoride, it may be desired to have a pH slightly lower than typical dentifrices. The pH is typically measured using a ratio of 1:3 of paste:water, whereby 1 gram of the oral care composition (e.g., toothpaste) is mixed into 3 grams of deionized water, and then the pH is assessed with a industry accepted pH probe that is calibrated under ambient conditions. The pH is measured by a pH meter with Automatic Temperature Compensating (ATC) probe. The pH meter is capable of reading to 0.001 pH unit.

After each usage the electrode should be washed free from the sample solution with water. Remove any excess water by wiping with a tissue, such as Kimwipes or equivalent. When electrode is not in use, keep electrode tip immersed in pH 7 buffer solution or electrode storage solution. Equipment details are as follows:
pH Meter: Meter capable of reading to 0.01 or 0.001 pH units.
Electrode: Orion Ross Sure-Flow combination: Glass body—VWR #34104-834/Orion #8172BN or VWR#10010-772/Orion #8172BNWP.
   Epoxy body—VWR #34104-830/Orion #8165BN or VWR#10010-770/Orion #8165BNWP.
   Semi-micro, epoxy body—VWR #34104-837/Orion #8175BN or VWR#10010-774/Orion #3175BNWP.
   Orion PerpHect combination: VWR #34104-843/Orion #8203BN semi-micro, glass body.
ATC Probe: Fisher Scientific, Cat. #13-620-16.

### pH Buffering Agent

The oral care compositions herein may include an effective amount of a buffering agent or pH trimming agents, as used herein, refer to agents that can be used to adjust the pH of the oral care compositions to the above-identified pH range. The buffering agents include alkali metal hydroxides, ammonium hydroxide, organic ammonium compounds, carbonates, sesquicarbonates, borates, silicates, phosphates, imidazole, and mixtures thereof.

Specific buffering agents include monosodium phosphate (monobasic sodium phosphate), trisodium phosphate (sodium phosphate tribasic dodecahydrate or TSP), sodium benzoate, benzoic acid, sodium hydroxide, potassium hydroxide, alkali metal carbonate salts, sodium carbonate, imidazole, pyrophosphate salts, sodium gluconate, lactic acid, sodium lactate, citric acid, sodium citrate, phosphoric acid.

In one embodiment, 0.01% to 3%, preferably from 0.1% to 1% of TSP by weight of the composition, and 0.001% to 2%, preferably from 0.01% to 0.3% of monosodium phosphate by weight of the composition is used. Without wishing to be bound by theory, TSP and monosodium phosphate may have calcium ion chelating activity and therefore provide some monofluorophosphate stabilization (in those formulations containing monoflurophospahte).

### Water

Water is commonly used as a carrier material in oral care compositions due to its many benefits. For example, water is useful as a processing aid, is benign to the oral cavity and assists in quick foaming of toothpastes. Water may be added as an ingredient in its own right or it may be present as a carrier in other common raw materials such as, for example, sorbitol and sodium lauryl sulphate.

The oral care compositions herein may include from 10% to 70%, or preferably from 15% to 30%, by weight of the composition, of total water content. The term “total water content” as used herein means the total amount of water present in the oral care composition, whether added separately or as a solvent or carrier for other raw materials but excluding that which may be present as water of crystallization in certain inorganic salts. Preferably, the water is USP water.

### Flavoring Composition

The oral care composition herein may include from 0.01% to 5%, preferably from 0.1% to 2%, by weight of the oral care composition, of a flavor composition. Examples of suitable flavoring agent that may be used in the flavoring composition include those described in U.S. Pat. No. 8,691,190; Haught, J. C., at paragraphs 39 and 40 to 45. Preferably, the flavor composition comprises:
(i) a flavor mixture comprising from greater than 0% to less than 55%, or from greater than 65% to 95%, by weight of the flavor composition, of methyl salicylcate; from greater than 0% to less than 30%, or from greater than 35% to 65%, by weight of the flavor composition, of menthol; from greater than 0% to less than 1%, or from greater than 5% to 50%, by weight of the flavor composition, of eugenol; and from greater than 0% to less than 3%, or from greater than 8% to 30%, by weight of the flavor composition, of cineol; or
(ii) a flavor mixture that is free or substantially free of methyl salicyclate, menthol, eugenol and cineol.

### Sweetener

The oral care compositions herein may include a sweetening agent. The sweetening agent is generally present in the oral care compositions at levels of from 0.005% to 5%, by weight of the composition. Suitable examples of sweetener include saccharin, dextrose, sucrose, lactose, xylitol, maltose, levulose, aspartame, sodium cyclamate, D-tryptophan, dihydrochalcones, acesulfame, sucralose, neotame, and mixtures thereof. Other suitable examples of sweetener are described in U.S. Pat. No. 8,691,190; Haught, J. C.

### Coloring Agents

The oral care compositions herein may include a coloring agent present in the amount of from 0.001% to 0.01%, by weight of the compositions. The coloring agent may be in the form of an aqueous solution, preferably 1% coloring agent in a solution of water. Suitable examples of coloring agents may include pigments, pealing agents, filler powders, talc, mica, magnesium carbonate, calcium carbonate, bismuth oxychloride, zinc oxide, and other materials capable of creating a visual change to the oral care compositions. Other suitable examples may include titanium dioxide (TiO₂). Titanium dioxide is a white powder which adds opacity to the compositions and is generally present in the oral care compositions at levels of from 0.25% to 5%, by weight of the composition.

### Surfactant

The oral care compositions herein may include a surfactant present in the amount of from 0.1% to 50%, from 0.025% to 9%, from 0.05% to 5%, from 0.1% to 2.5%, from 0.5% to 2%, or from 0.1%, to 1%, by weight of the compositions. The surfactant may be selected from anionic, nonionic, amphoteric, zwitterionic, cationic or mixtures thereof. Suitable examples of anionic surfactants may include those described in U.S. Pat. No. 8,691,190; Haught, J. C., at paragraphs 32, 33, 34 and 35. Suitable examples of zwitterionic or amphoteric surfactants are described in U.S. Pat. No. 8,691,190; Haught, J. C., at paragraph 36, cationic surfactant at paragraph 37, and nonionic surfactants at paragraph 38.

### Humectants

The oral care compositions herein may include humectants present in the amount of from 0% to 70%, or from 15% to 55%, by weight of the compositions. Humectants keep oral care compositions from hardening upon exposure to air and certain humectants may also impart desirable sweetness of flavor to dentifrice compositions. Suitable examples of humectants may include glycerin, sorbitol, polyethylene glycol, propylene glycol, xylitol, trimethyl glycine, and mixtures thereof. Other examples may include other edible polyhydric alcohols.

### Abrasives

The oral care compositions herein may further include one or more abrasives present in the amount of from 0.1% to 60%, or from 1% to 50%, or from 2% to 40%, or from 4% to 30%, by weight of the composition. Suitable examples may include precipitated silica, fused silica, calcium carbonate, dicalcium phosphate dihydrate, phosphates (including orthophosphates), pyrophosphates, perlite, pumice, nanodiamonds, surface treated and de-hydrated precipitated silica, rice hull silica, silica gels, aluminas, polymetaphosphates, other inorganic particulates, and mixtures thereof. Other examples of abrasive materials useful herein may include dicalcium orthophosphate dihydrate, calcium pyrophosphate, tricalcium phosphate, calcium polymetaphosphate, insoluble sodium polymetaphosphate, hydrated alumina, beta calcium pyrophosphate, calcium carbonate, and resinous abrasive materials such as particulate condensation products of urea and formaldehyde, and others such as disclosed in U.S. Pat. No. 3,070,510; Cooley, W. E.

### Anti-Sensitivity Agent

The oral care compositions herein may include an anti-sensitivity agent present in the amount of from 0.001% to 20%, or from 0.1% to 5%, by weight of the compositions. Suitable examples of anti-sensitivity agent may include those described in U.S. Pat. No. 8,926,949; Dayanim, R., at paragraph 41, and U.S. Patent Publication No. 2009/0311200; Lambert, P., at paragraph 59.

### Whitening and Oxidizing Agent

The oral care compositions herein may include a whitening or oxidizing agent present in the amount of from 0.01% to 30%, or from 0.1% to 10%, or from 0.5% to 5%, by weight of the compositions. Suitable examples may include hydrogen peroxide, urea peroxide, calcium peroxide, sodium peroxide, zinc peroxide, or combinations thereof. Other examples are those described in U.S. Pat. No. 8,691,190; Haught, J. C., at paragraph 56.

### Anti-Inflammatory Agent

The oral care compositions herein may include an effective amount of an anti-inflammatory agent. Suitable examples may include those described in U.S. Patent Publication No. 2011/0104081, Scott, D. C., at paragraph 55.

### Anti-Calculus Agent

The oral care compositions herein may include an anti-calculus agent present in the amount of from 0.05% to 50%, or from 0.05% to 25%, or from 0.1% to 15%, by weight of the compositions. Suitable examples may include those described in U.S. Patent Publication No. 2011/0104081, Scott, D. C., at paragraph 64, and U.S. Patent Publication No. 2012/0014883; Scott, D. G., at paragraphs 63 to 68.

### Chelating Agent

The oral care compositions herein may include an effective amount of a chelating agent, also referred to as sequestrants, many of which also have anti-calculus activity or tooth substantive activity. Use of chelating agents in oral care products is advantageous for their ability to complex calcium such as found in the cell walls of bacteria, to disrupt plaque and to complex with metallic ions. Chelation of ions, such as iron or copper, helps retard oxidative deterioration of finished products. Suitable examples of chelating agent may include those described in U.S. Patent Publication No. 2011/0020246; Strand, R., at paragraphs 21 to 28.

### Tooth Substantive Agent

The oral care compositions herein may include an effective amount of a tooth substantive agent. For purposes of this application, tooth substantive agents are included as chelants also. Suitable examples may include polymeric surface active agents (“PMSAs”), including polyelectrolytes, more specifically anionic polymers. Other examples may include those described in U.S. Patent Publication No. 2012/0014883; Scott, D. G., at paragraphs 74 to 84.

### Analgesic and Anesthetic Agent

The oral care compositions herein may include an effective amount of an analgesic or desensitizing agent. Suitable examples may include those described in U.S. Pat. No. 9,005,585; Deckner, G. E., at paragraph 117.

### Other Ingredients

The present oral care composition can comprise the usual and conventional ancillary components that are known to one skilled in the art. It will be appreciated that selected components for the oral care compositions must be chemically and physically compatible with one another.

### Method of Use

In one aspect, the present invention relates to a method for cleaning or polishing teeth in a human subject. The method of cleaning or polishing herein comprises contacting a subject's teeth with the oral care compositions according to the present invention.

In another aspect, the present invention also relates to a method of promoting Gum Health in a human subject comprising administering to the subject's oral cavity an oral care composition according to the present invention. Preferably, the method of promoting Gum Health occurs at least within a period selected from the group consisting of:
a) from time 0 hours to 72 hours;
b) from time 0 hours to 48 hours;
c) from time 0 hours to 24 hours;
   wherein time 0 hours is upon administration of the oral care composition according to the present invention.

In yet another aspect, the present invention also relates to a method of promoting Gum Health, wherein Gum Health is selected from:
(i) improving gingival wound healing in the oral cavity;
(ii) improve reduction of bacterial activity in the oral cavity; or
(iii) combination thereof.

The methods as described above may be by brushing (e.g., toothbrushing) with an oral care composition (e.g., dentrifice) or rinsing with an oral care composition (e.g., dentifrice slurry or mouthrinse). The oral care compositions may be applied neat or via a delivery apparatus such as, for example, a toothbrush. Other methods include contacting the topical oral gel, mouthspray, toothpaste, dentifrice, tooth gel, tooth powders, tablets, subgingival gel, foam, mouse, chewing gum, lipstick, sponge, floss, petrolatum gel, or denture product or other form with the subject's teeth and oral mucosa. Depending on the embodiment, the oral care composition may be used as frequently as toothpaste, or may be used less often, for example, weekly, or used by a professional in the form of a prophy paste or other intensive treatment.

### EXAMPLES

The following examples and descriptions further clarify embodiments within the scope of the present invention. These examples are given solely for the purpose of illustration and are not to be construed as limitations of the present invention as many variations thereof are possible without departing from the spirit and scope.

### A: Examples 1 to 10

Examples 1 to 10 are dentifrice compositions shown below with amounts of components in wt %. They may be suitably prepared by conventional methods chosen by the formulator.

Examples 1 to 6 are inventive formulations according to the present invention, made with a stannous ion source (e.g., stannous chloride) and a single antifibrinolytic agent (e.g., TA, EACA, or PAMBA) at two concentrations, respectively. Example 7 is an inventive formulation made with stannous chloride and two antifibrinolytic agents (e.g., TA and EACA). In parallel, control formulations examples 8-10, are prepared. Example 8 is made without the stannous ion source, Example 9 is made without the antifibrinolytic agent, and Example 10 is made without either components. All of the compositions are prepared by admixture of the components in Tables 1 and 2, in the proportions indicated.

TABLE 1
Inventive Compositions Examples 1 to 7

| | Amount (Wt %) | | | | | | |
|---|---|---|---|---|---|---|---|
| Ingredients | Ex. 1 | Ex. 2 | Ex. 3 | Ex. 4 | Ex. 5 | Ex. 6 | Ex.7 |
| Sorbitol Solution 70% | 48.000 | 38.00 | 48.000 | 48.000 | 38.000 | — | 38.000 |
| (Archer Daniels Midland) | | | | | | | |
| Sodium Fluoride | 0.21 | 0.321 | 0.210 | 0.210 | 0.321 | — | 0.321 |
| Tranexamic Acid (TA) | 0.27 | 0.50 | — | — | — | 0.500 | 0.500 |
| epsilon aminocaproic acid | — | — | 0.270 | — | — | — | 0.500 |
| (EACA) | | | | | | | |
| p-aminomethylbenzoic acid | — | — | — | 0.270 | 0.500 | — | — |
| (PAMBA) | | | | | | | |
| Glycerin | — | — | — | — | — | 35.500 | — |
| Propylene Glycol | — | — | — | — | — | 7.000 | — |
| PEG-6 | — | — | — | — | — | 7.000 | — |
| Sodium Polyphosphate | — | — | — | — | — | 13.000 | — |
| Trosodium Phosphate | — | — | — | — | — | 1.100 | — |
| Dodecahydrate | | | | | | | |
| Stannous Fluoride | — | — | — | — | — | 0.454 | — |
| Zinc Lactate Dihydrate | — | — | — | — | — | 1.900 | — |
| Zinc Citrate | 0.533 | 0.533 | 0.533 | 0.533 | 0.533 | — | 0.533 |
| Stannous Chloride | 1.160 | 1.160 | 1.160 | 1.160 | 1.160 | 0.462 | 1.160 |
| Dihydrate | | | | | | | |
| Sodium Gluconate | 1.064 | 1.064 | 1.064 | 1.064 | 1.064 | 1.099 | 1.064 |
| Xanthan Gum | 0.875 | 0.875 | 0.875 | 0.875 | 0.875 | 0.250 | 0.875 |
| Carrageenan Mixture Iota | 1.500 | 1.500 | 1.500 | 1.500 | 1.500 | 0.600 | 1.500 |
| Silica | | | | | | | |
| Silica Abrasive | 16.000 | 16.000 | 16.000 | 16.000 | 16.000 | 25.000 | 16.000 |
| Thickening Silica | — | — | — | — | — | 0.750 | — |
| Sodium Lauryl Sulfate | 5.000 | 7.500 | 5.000 | 5.000 | 7.500 | 3.400 | 7.500 |
| (28% soln.) | | | | | | | |
| Sodium Saccharin | 0.300 | 0.250 | 0.300 | 0.300 | 0.250 | 0.700 | 0.250 |
| Flavor/sensate oils | 1.100 | 1.100 | 1.100 | 1.100 | 1.100 | 1.200 | 1.100 |
| Sodium Hydroxide | 0.980 | 0.980 | 0.980 | 0.980 | 0.980 | — | 0.980 |
| Water and minors | q.s. | q.s. | q.s. | q.s. | q.s. | qs | q.s. |
| (e.g., color soln.) | | | | | | | |
| Total | 100% | 100% | 100% | 100% | 100% | 100% | 100% |
| Target pH | 6 | 6 | 6 | 6 | 6 | 5.7 | 6 |

TABLE 2
Control Compositions Examples 8 to 10

| | Amount (Wt %) | | |
|---|---|---|---|
| Ingredients | Ex. 8 | Ex. 9 | Ex. 10 |
| Sorbitol Solution 70% | 48.000 | 48.000 | 48.000 |
| Sodium Fluoride | 0.321 | 0.321 | 0.321 |
| Tranexamic Acid | 0.500 | — | — |
| Zinc Citrate | — | 0.533 | — |
| Stannous Chloride Dihydrate | — | 1.160 | — |
| Sodium Gluconate | 1.064 | 1.064 | 1.064 |
| Xanthan Gum | 0.875 | 0.875 | 0.875 |
| Carrageenan Mixture | 1.500 | 1.500 | 1.500 |
| Iota Silica | | | |
| Silica Abrasive | 16.000 | 16.000 | 16.000 |
| Sodium Lauryl Sulfate | 5.000 | 5.000 | 5.000 |
| (28% soln.) | | | |
| Sodium Saccahrin | 0.300 | 0.300 | 0.300 |
| Flavor | 1.100 | 1.100 | 1.100 |
| Sodium Hydroxide | 0.980 | 0.980 | 0.980 |
| Water and minors | q.s. | q.s. | q.s. |
| (e.g., color soln.) | | | |
| Total | 100% | 100% | 100% |
| Target pH | 6.0 | 6.0 | 6.0 |

### Example 11—Assay for Measuring Improve Gingival Wound Healing in the Oral Cavity

The following assay is used to determine improved gingival wound healing in the oral cavity for the oral care compositions of the present invention and controls. The assay involves gentle probing of gingival crevice to assess presence or absence of bleeding. Gingivitis is assessed according to the Mazza modification of the Papillary Bleeding Index (“PBI”, and according to Muhlemann, H. R.: J. Prev. Dent. 1977; 4:6), otherwise referred to as the “Mazza Index” to determine the number of bleeding sites (as defined by Mazza, 1981). For this measurement, probing is done on the mesiofacial and the distolingual surface of each tooth, at a maximum of 56 sites. The probe is placed in the gingival sulcus to a depth of approximately 0.5 mm to 1.0 mm and swept along the soft tissue aspect of the sulcus from its insertion point to the tip of the interdental papilla. All facial or lingual surfaces of each quadrant are swept before measurements are made. The measurements are made beginning with the first tooth swept.

Oral soft tissues and gingivitis examination using the Mazza Index are conducted at Baseline (i.e., 0 hr), Day 3 and Week 4. The Mazza Index is defined as in Table 3.

TABLE 3
Mazza Index for Measuring Gingival Bleeding

| Score | Description |
|---|---|
| 0 | Normal appearing gingival (i.e., no color change), |
| | and no bleeding upon probing. |
| 1 | Color change related to inflammation, |
| | but no bleeding on probing. |
| 2 | Slight bleeding at the point of probing. |
| 3 | Bleeding extending from the point of |
| | probing and flowing around the gingival margin. |
| 4 | Profuse bleeding that overflows the gingival margin. |
| 5 | Spontaneous bleeding without probing. |

Experiments are conducted at Procter & Gamble (Beijing) Technology Co., Ltd. Oral Care Department, with approval from the P&G Beijing Technical Center (China) Institutional Review Board and in accordance with the World Medical Association Declaration of Helsinki (1996 amendment). ICH Guidelines for Good Clinical Practice (“GCP”) are followed.

To start, subjects who met the study entrance requirement are randomly assigned to treatment or control groups balanced on Baseline Mazza Index scores using a SAS randomization program. Individuals meeting the following criteria are included: be at least 18 years of age; possess a minimum of 12 natural anterior teeth; have at least 5 bleeding sites as measured by Mazza Index at initial visit (i.e., Baseline); have gingivitis but not periodontitis; be in good general health as determined by the Investigator/designee based on a review of the medical history/update for participation in the study. Exclusion criteria for individuals includes: severe periodontal disease, as characterized by purulent exudates, generalized mobility, and/or severe recession; any condition which requires antibiotic premedication for the administration of a dental prophylaxis; self-reported pregnancy or intent to become pregnant during the course of the study and nursing females; atypical discoloration or pigmentation in the gingival tissue; fixed facial orthodontic appliances; atypical discoloration or pigmentation in the gingival tissue; use of antibiotics any time during the study; any diseases or conditions that could be expected to interfere with the subject safely completing the study. Clinical parameters for each subject are monitored across the whole study. Individuals that fell into the exclusion criteria are excluded from study participation.

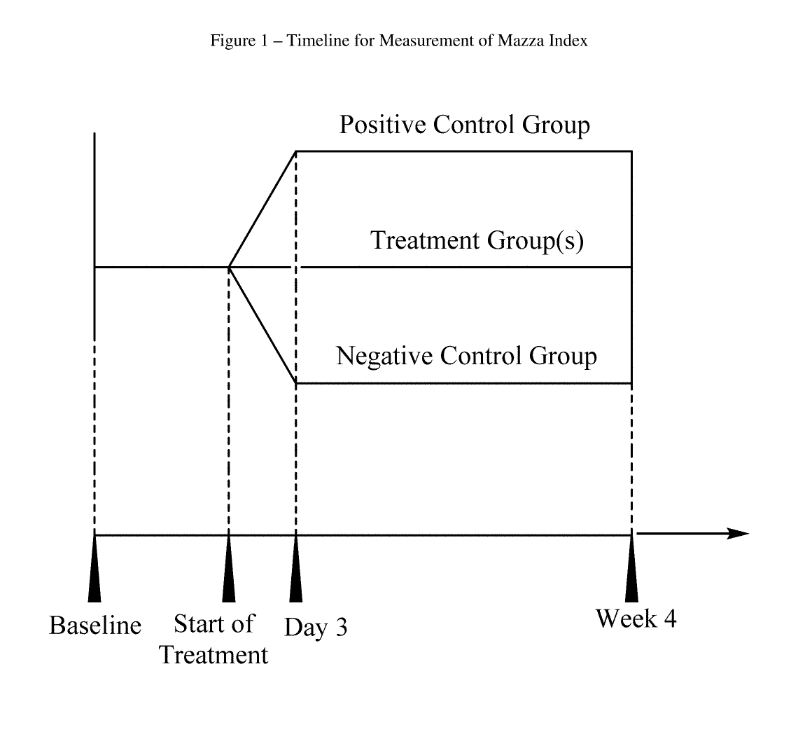

Study is 4-week, double-blind and parallel groups. Subjects are treated with products from: the negative control group, the positive control group, and the treatment (i.e., inventive compositions) group. The negative control group receives a regular fluoridated toothpaste product (e.g., 0.321% sodium fluoride—Crest Cavity Protection Product, Lot Number 60771864AA, 2018.03.17) (“CCP Product”). The positive control group receives a marketed traditional Chinese Medicine and TA formulation Yun Nan Bai Yao Liu Lan product, Lot Number 20190408 F05 (“YNBY Product”). The treatment groups received any one of the Inventive Compositions as disclosed in Examples 1-7, which contains a stannous chloride and one or more antifibrinolytic agent(s). Each of the control or treatment legs are applied topically to the teeth/gingival surface twice daily for period of time of 4 weeks. Mazza Index assessments are performed on the subjects to score for reduction of bleeding sites at Baseline, Day 3 and at 4 weeks (i.e., end of the study), as shown in FIG. 1. The data are analyzed with analysis of covariance (“ANCOVA”), with the respective Baseline as the model covariate using the Statistical Analysis System (“SAS”).

Results:

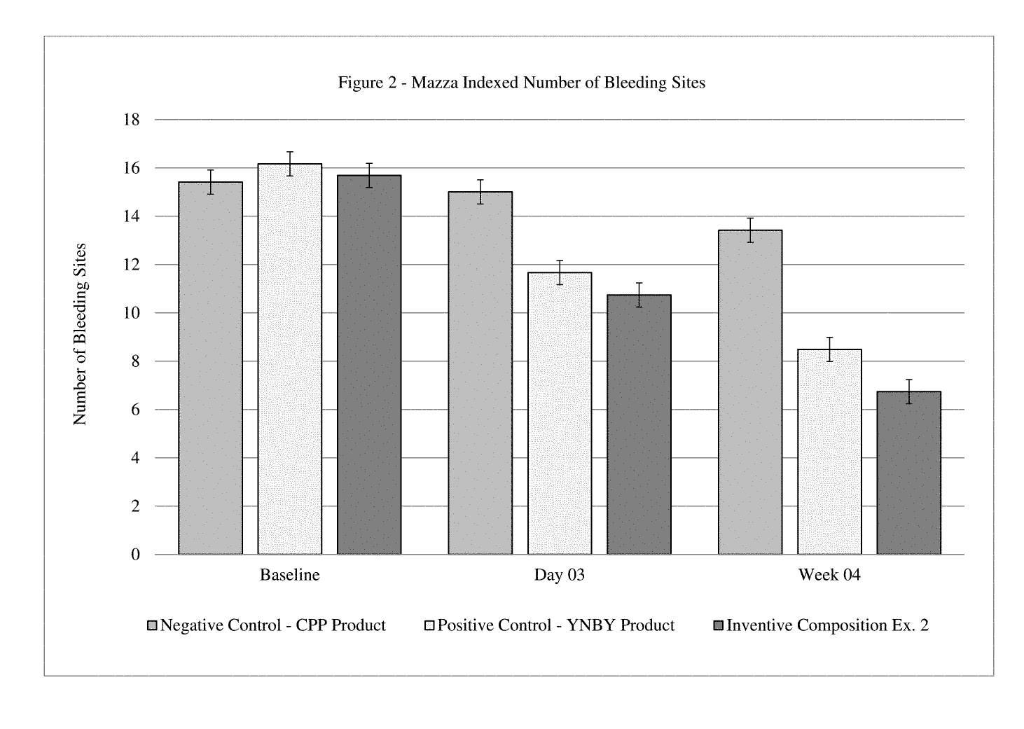

150 qualified subjects completed the study. The treatment group received Inventive Composition Example 2. The control groups and treatment group are well balanced on age and gender. Baseline means are balanced between the control groups and treatment group (p>0.43). The results in terms of the number of bleeding sites, based on the Mazza Index, of the control groups and treatment group are averaged (as well as their confidence intervals) and then plotted as bar graphs, as shown in FIG. 2, to enable the comparisons between control groups and the treatment group. The p-values for the product differences are obtained with p<0.01.

FIG. 2 shows the effect of the Sn and TA on the reduction of gingival bleeding of the Inventive Composition Ex. 2. With reference to FIG. 2, the Inventive Composition Ex. 2 (containing both TA and Sn) has an average of 10.7 bleeding sites at Day 3, and 6.73 bleeding sites at Week 4, vs. the positive control YNBY Product has an average of 11.67 bleeding sites at Day 3 and 8.49 bleeding sites at Week 4. The negative control CCP Product, which contains no Sn and no TA, has an average of 15.01 bleeding sites at Day 3 and 13.41 bleeding sites at Week 4. The results show that the addition of the Sn to the TA in the Inventive Composition Ex. 2 provides a greater improvement of the reduction of the number of bleeding sites (i.e., approx. −10 bleeding sites) between Baseline and Week 4, while the positive control YNBY Product having only TA has a smaller reduction (i.e., approx. −5 bleeding sites) in the number of bleeding sites over the same timeframe. The negative control CCP Product, which contains no Sn and no TA, shows only modest reduction (i.e., approx. −2 bleeding sites) in the number of bleeding sites over the 4-week study.

The results demonstrate that the combination of an antifibrinolytic agent and an anti-bacterial agent (e.g., stannous ion source) provides enhanced gingival anti-bleeding efficacy. In fact, the Inventive Composition Ex. 2 demonstrates efficacy at lower levels of antifibrinolytic agents (i.e., 0.50% TA). This is an advantageous since certain antifibrinolytic agents (e.g., TA, EACA, etc.) while efficacious are known to cause discoloration and instability issues with the oral care compositions particularly at higher concentrations that have been employed to provide efficacy. Thus, by using lower levels, the discoloration and instability will both be avoided.

### B: Assay for Measuring Improve Penetration of Anti-Bacterial Agent in the Biofilms

In order to determine improve penetration of anti-bacterial agent in the biofilms, the following assay is used to assess co-localization percentage of stannous ions with bacteria via in situ plaque biofilms for inventive oral care compositions of the present invention and controls. Details of the assay are described below.

(a) Substrate for Biofilm Growth

Hydroxyapatite (“HA”) disks are used for in situ growth of biofilms. The HA disks are designed having three parallel grooves (i.e., 200 μm wide; 200 μm deep for two sides' grooves; while 500 μm wide and 500 μm deep for the middle groove) in each disk. When attaching disks to subject's mouth, keep these grooves vertical, to mimic interproximal gap between teeth, which is the hard-to-clean area where plaque generally tends to accumulate. This model allows the collection of undisturbed plaque from the grooves. HA disks are manufactured by Shanghai Bei′erkang biomedicine limited company.

(b) Wearing the Splint

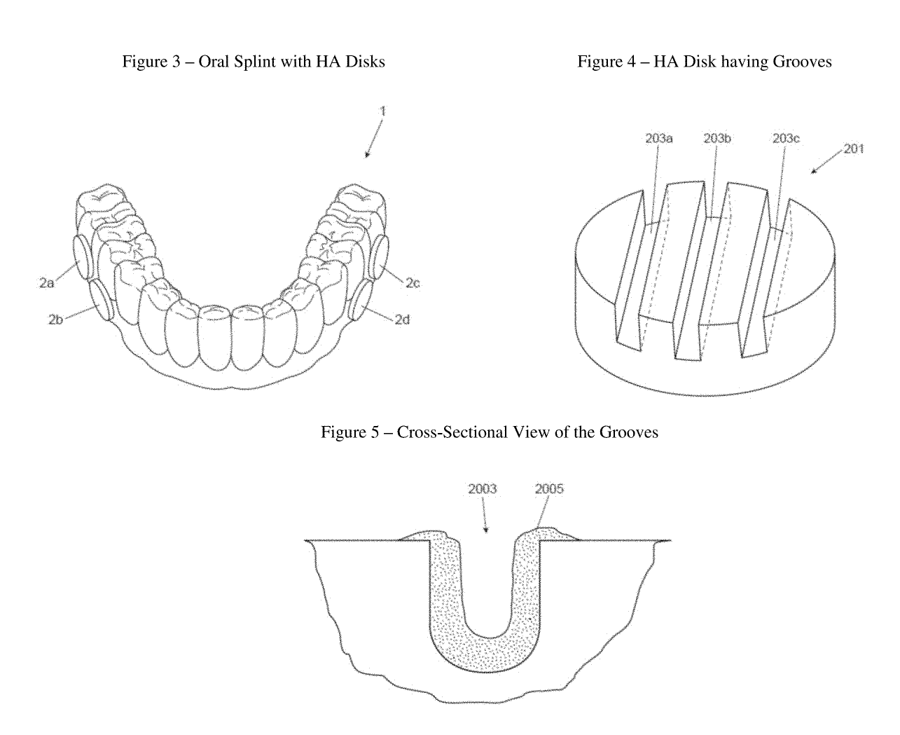

Human subjects wear the splint. Each subject wears up to 12 HA disks on the splint to ensure that, at least, 9 HA disks are available after 48 hours. A non-limiting example of such a splint and HA disks are shown in FIG. 3. With reference to FIG. 3, the device (1) holds a plurality of HA disks (2a-2d). In a specific example, and with reference to FIG. 4, the HA disk (201) has three parallel grooves (203) (the two sides' grooves (203a and 203c) are 300 μm wide and 300 μm deep; while the middle grove (203b) (in between the two side grooves) is 500 μm wide and 500 μm deep). The middle groove is designed wider and deeper than the two sides' grooves so that the HA disk can be more easily separated into two identical half-disks for head-to-head comparison purposes. FIG. 5 is a schematic of a cross-sectional view of the groove (2003) with biofilm (2005) therein. Further details of the HA disks are described in PCT patent application no. PCT/CN2015/089238, with the international filing date of Sep. 9, 2015.

Although not shown in FIG. 5, the disks can be positioned such that the recede is in the inter-dental space between the teeth (since this location is prone to plaque (given the difficulty in cleaning, etc.)). The subjects withdraw the splint only during meals (the splint stored in an opaque container in humid conditions) and to perform oral hygiene procedures Immediately thereafter, the splint is worn again. Subjects are asked to use a straw when drinking.

(c) In-Situ Biofilms Release from HA Desk

All HA disks are removed from the splint at 48 hours by tweezers. Tweezers are used to hold the edge of HA chips and transfer the HA disk to a 2 mL centrifuge tube containing PBS (phosphate buffered saline) solution. Tweezers are washed thoroughly (water; 75% alcohol; and then deionized water) before every disk transfer.

(d) Preparation of Toothpaste Supernatent

15 grams of deionized water is added to 5 grams toothpaste (using any one of the inventive Composition Examples 1-7, control Composition Examples 8-10 or marketed formulations (e.g., Traditional Chinese Medicine and TA formulation Yun Nan Bai Yao Liu Lan product, Lot Number 20190408 F05 (“YNBY Product”). After stirring thoroughly, the mixture is centrifuge at 12,000 RPM for 20 minutes. The supernatant is prepared one day before usage and stored at 4° C.

(e) Confocal Laser Scanning Microscopy

After the HA disks are removed from the splint. The HA disks are used for ex vivo treatment by the different inventive and control compositions. After being treated with the subject supernatant and labeled with microbial fluorescent probe and stannous fluorescent probe (such as described in PCT Publication No. WO2015/139263; Shi et al.), the biofilms in the grooves are measured by confocal laser scanning microscopy (“CLSM”) (as described below). Preferably, the stannous fluorescent probe is tert-butoxy-carboxamide,N-3′,6′-bis(diethylamino)-3-oxospiro[1H-isoindole-1,9′-[9H]xanthen]-H)-yl] (available from Dr. Tao Yu, Fudan University, Shanghai, China). Preferably, the microbial fluorescent probe is the Molecular Probes™ LIVE/DEAD® BacLight™ system (available from Thermo Fisher).

(f) Disk Preparation

The HA disks are rinsed in PBS solution and each HA disk is divided into two halves by tweezers. Thereafter, each half-disk is placed into 500-1000 μL of PBS solution statically for 1 minute. Each disk is treated for two minutes by either PBS solution or toothpaste supernatant. Each disk is washed by holding each disk with tweezers, shaken for ten rounds of back and forth in 1 mL of PBS solution, and then this washing cycle is repeated. Then each disk is immersed into 500-1000 μL PBS solution statically for 5 minutes.

(g) Fluorescence Staining and Microscopy

It is reported that the LIVE/DEAD® BacLight™ system is a reliable alternative when assessing bacterial vitality in a natural plaque biofilms, in which there are several types of bacteria present. The LIVE/DEAD® BacLight™ fluorescence assay stains the bacteria in red or green depending on the permeability of their membrane. The ratio of green/red is well correlated with live/dead ratio. It is considered that live/dead staining methods are reliable when analyzing antimicrobial agent activity. After treatment and immersing, each half-disk is stained with the Sn probe together with Syto-9 probe (containing 5 μM Syto-9 and 5 μM Sn probe) for 30 minutes in the dark and the other half-disk is stained with L7012 LIVE/DEAD® dye solution (containing 5 μM Syto-9+30 μM propidium iodide) for 15 minutes in the dark. After staining, each disk is immersed into 500-1000 μL PBS solution statically for 2 minutes. The disks are washed again, by holding each disk with tweezers, shaken for five rounds of back and forth in 1 mL PBS solution, and repeated. For L7012 LIVE/DEAD® dye stained samples, the following parameters are used: λex=488 nm, λem=500/635 nm, 20× objective lens, and scanning from bottom of surface bacteria for 60 μm with step size=3 μm. For SYTO-9/Sn dye stained samples, the following parameters are used: λex=488 nm/543 nm, λem=500/580 nm, 20× objective lens, and scanning from bottom of surface bacteria for 60 μm with step size=3 μm.

(h) Confocal Laser Scanning Microscopy

The Leica™ TCS SP8 AOBS spectral confocal microscope is used. The confocal system consists of a Leica™ DM6000B upright microscope and a Leica™ DMIRE2 inverted microscope. An upright stand is used for applications involving slide-mounted specimens; whereas the inverted stand, having a 37° C. incubation chamber and CO₂ enrichment accessories, provides for live cell applications. The microscopes share an exchangeable laser scan head and, in addition to their own electromotor-driven stages, a galvanometer-driven high precision Z-stage which facilitates rapid imaging in the focal (Z) plane. In addition to epifluorescence, the microscopes support a variety of transmitted light contrast methods including bright field, polarizing light and differential interference contrast, and are equipped with 5×, 20×, 40×, 63× (oil and dry) and 100× (oil) Leica™ objective lenses.

The laser scanning and detection system is described. The TCS SP2 AOBS confocal system is supplied with four lasers (one diode, one argon, and two helium neon lasers) thus allowing excitation of a broad range of fluorochromes within the UV, visible and far red ranges of the electromagnetic spectrum. The design of the laser scan head, which incorporates acousto-optical tunable filters (“AOTF”), an acousto-optical beam splitter (“AOBS”) and four prism spectrophotometer detectors, permits simultaneous excitation and detection of three fluorochromes. The upright microscope also has a transmission light detector making it possible to overlay a transmitted light image upon a fluorescence recording.

Leica™ Confocal software is used. The confocal is controlled via a standard Pentium PC equipped with dual monitors and running Leica™ Confocal Software. The Leica Confocal Software provides an interface for multi-dimensional image series acquisition, processing and analysis, that includes 3D reconstruction and measurement, physiological recording and analysis, time-lapse, fluorochrome co-localization, photo-bleaching techniques such as FRAP and FRET, spectral immixing and multicolour restoration. Regarding image analysis, the L7012 LIVE/DEAD® dye stained samples, are chosen to quantify ratio of red and green pixels, and for SYTO-9/Sn dye stained samples are chosen to quantify overlap efficiency of red and green pixels. Using the software, the pixel overlap of “green” bacterial probes and that of “red” stannous probes are identified, and then this value is divided by all non-black pixels (that include non-overlapping stannous probes) to provide a co-localization percentage of stannous in bacteria. Generally the higher this co-localization percentage, the more efficacious the oral care product is in delivering stannous into bacteria.

Results:

Subjects are treated with the Inventive Composition Ex. 2 (i.e., Sn+0.50% TA), Control Composition Ex. 9 (i.e., Sn only), and the marketed YNBY Product. The results are provided in Table 4.

TABLE 4
Biofilm Bacterial Activity and Active Penetration

| | | Sn Co- | Sn |
|---|---|---|---|
| | Bacterial | localization | Overlap |
| | Kill (%) | Rate (%) | Co-efficient |
| Inventive | 55.00 | 82.29 | 0.9301 |
| Composition | | | |
| Ex. 2 | | | |
| Control | 46.39 | 61.48 | 0.9206 |
| Composition | | | |
| Ex. 9 | | | |
| YNBY Product | 47.86 | 2.25 | 0 |
| PBS | 19.14 | 0 | 0 |

The results show that the Inventive Composition Ex. 2 containing the antifibrinolytic agent (e.g., tranexamic acid) and an anti-bacterial agent (e.g., stannous ion source) effectively improves the reduction of the bacterial activity (55.00% bacterial kill) vs. the lower bacterial kill (46.39%) for the Control Composition Ex. 10, which contains anti-bacterial agent and stannous but no tranexamic acid. The marketed YNBY Product shows comparable bacterial kill (47.86%) relative to the Control Composition Ex. 9.

The results demonstrate the markedly higher stannous co-localization percentage with the Inventive Composition Ex. 2 (82.29%) over the Control Composition Ex. 9 (61.48%) and the YNBY Product (2.25%). The results also show that TA does not impair the stannous releasing efficiency as measured through the Sn overlap co-efficient, where the Sn overlap coefficient for the Inventive Composition Ex. 2 (0.9301) and the Control Composition Ex. 9 (0.9206) are substantially similar. In effect this data supports the improve penetration of the stannous ion into the biofilms when combined with an antifibrinolytic agent (e.g., tranexamic acid) over the control and marketed formulations.

### C: Assay for Measuring Improved Wound Healing of Human Gingival Fibroblasts

In-vitro human gingival fibroblasts are used to assess the effects of wound healing migration as a result of treatment with Inventive Compositions and Control Compositions. The method involves three stages:

Stage 1—Culturing Primary Human Gingival Fibroblasts (“HGF”)

Human gingival fibroblasts are collected from tooth extraction patients and washed with 5 mL of PBS (phosphate buffered saline). The tissues are chopped into small pieces and placed into 15 mL centrifuge tube. The samples are digested with equal amounts of 1 mL 8% dispase and 1 mL 6% collagenase for 1 hr at 37° C., during which time the samples need to be shaken every 15 minutes. Once the digestion process is complete, the tube is centrifuged at 1100 RPM for 6 minutes at room temperature. After centrifugation, a pellet of cells is formed in the bottom of the tube separating them from the supernatant solution. Then the supernatant is discarded and the cell pellet is suspended in 3 mL of fresh Minimum Essential Medium (“MEM”, available from Thermo Fisher) culture media then transferred to a petri dish. The petri dish with cells are placed in the incubator at 37° C. with 5% CO₂ for about 10 days. The petri dish is checked for changes in media color every two days. Fresh culture media is replaced if changes in media color occurred.

Stage 2—Sub-Culturing Human Gingival Fibroblast

When there is 80-90% cell monolayer coverage of the petri dish, then the present culture media is removed and washed with 5 mL of PBS. 1 mL 0.25% trypsin-EDTA solution is added and the cells sit for about 1-2 minutes at 37° C. until the cells are visibly round-shaped. It may be necessary to tap the petri dish to remove any sticky cells from the petri dish surface. At least 1 mL of fresh MEM culture media is added to inactivate the trypsin and the cells are collected into a 15 mL centrifuge tube. The tube is then centrifuged at 1100 RPM for 6 minutes at room temperature. The supernatant is discarded and cell pellet is re-suspended in 4 mL of fresh MEM culture media in the same centrifuge tube. 4 petri dishes are each placed with 1 mL cell suspension and 9 mL fresh MEM culture media in the incubator at 37° C. with 5% CO₂ for about 3-5 days until 80-90% cell monolayer coverage on the petri dishes are observed. This stage should be repeated 2-4 times before the wound healing assay to achieve the highest cell viability.

Stage 3—Wound Healing Assay

When there is 80-90% cell monolayer coverage on the petri dishes, the present culture media is removed and washed with 5 mL of PBS. 1 mL 0.25% trypsin-EDTA solution is added and the cells sit for about 1-2 minute at 37° C. until the cells are visibly round-shaped. It may be necessary to tap the culture petri dish to remove any sticky cells from the petri dish surface. At least 1 mL of fresh MEM culture media is added to inactivate the trypsin and the cells are collected into a 15 mL centrifuge tube. The tube is then centrifuged at 1100 RPM for 6 minutes at room temperature. The supernatant is discarded and cell pellet is re-suspended in 6 mL of fresh MEM culture media. 1 mL cell suspension and 1 mL fresh MEM culture media are respectively added into each well of a 6-well plate. The plates are incubated at 37° C. with 5% CO₂ until 50-70% cell monolayer coverage is formed. The outer bottoms of wells are then marked with a line in middle as the reference line during image acquisition. A wound is created manually by scraping the right half of cell monolayer with a sterilized 1 mL pipette tip. The cells are washed with 2 mL PBS to remove any suspended cells until no suspended cells are visible. 2 mL culture media, and 2 ml culture media containing 1% Control Compositions or 2 mL culture media containing 1% Inventive Compositions are added to the wells.

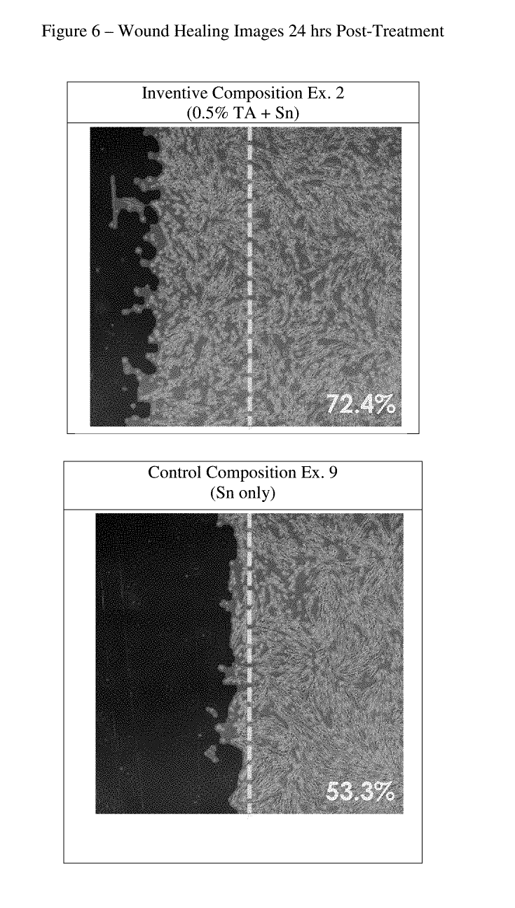

High density digital images of the HGF are captured with an Olympus® IX71 digital SLR camera with an Olympus® UIS2 WHN10× objective lens. The first images are acquired at time 0 hr (i.e., Baseline) by using the middle line markings on the plates as a reference line. The plates are then incubated at 37° C. with 5% CO₂ for varying time intervals as described below. The matched photographed region is acquired as previously, and images are acquired at later time intervals (e.g., 16 hrs, 24 hrs, 48 hr, 65 hrs, 72 hrs, etc.) after baseline to assess the cell coverage (%) as an indication of the wound healing performance under the different treatment legs. Images are evaluated by Wimasis® WimScratch software (available from Wimasis GmbH, Germany) to determine the degree (i.e., percentage) of HGF cell coverage (i.e., wound healing) pass the marked wound boundary, as indicated by the dotted line, as compared to the matching baseline image for each sample. WimScratch software utilizes advanced edge detection and overlay techniques to recognize cells and blank area, i.e. the green overlay in the image (shown as grey area in FIG. 6) represents the cell-covered area of the particular image and the grey area (shown as black area in FIG. 6) represents the wound area. The readout is presented for both area and is normalized as percent of total area.

Results:

FIG. 6 shows images of HGF 24 hrs post-treatment with the Inventive Composition Ex. 2 (i.e., Sn+0.50% TA) or the Control Composition Ex. 9 (i.e., Sn only). With reference to FIG. 6, the results show that the Inventive Composition Ex. 2 containing the antifibrinolytic agent and an anti-bacterial agent (e.g., stannous ion source) effectively improves the wound healing through increased cell coverage (22.4%=72.4% total coverage−50% baseline) post the marked wound (marked by the dotted line) relative to the lower cell coverage (3.3%=53.3% total coverage−50% baseline) for the HGF treated with the Control Composition Ex. 9.

### D: Mouth Rinse Compositions

Mouth rinse compositions according to the present invention are shown below as Examples 11-14 in Table 5. These compositions contain a stannous ion source and an antifibrinolytic agent. Preferably, these compositions exhibit improved Gum Health benefits versus commercially available formulations without these ingredients.

TABLE 5
Mouth Rinse Formulations

| | Amount (Wt %) | | | |
|---|---|---|---|---|
| Ingredients | Ex. 11 | Ex. 12 | Ex. 13 | Ex. 14 |
| Glycerin | 5.000 | 5.000 | 5.000% | 5.000 |
| Stannous Chloride | 0.116 | 0.116 | 0.116 | 0.116 |
| Dihydrate | | | | |
| Tranexamic Acid | 0.05 | 0.500 | — | — |
| (“TA”) | | | | |
| epsilon aminocaproic | — | — | 0.500 | — |
| acid (“EACA”) | | | | |
| p-aminomethylbenzoic | — | — | 0.500 | — |
| acid (“PAMBA”) | | | | |
| Sucralose | 0.030 | 0.030 | 0.030 | 0.030 |
| Ethanol | 5.000 | 5.000 | 5.000 | 5.000 |
| Methyl Paraben | 0.020 | 0.020 | 0.020 | 0.020 |
| Propyl Paraben | 0.005 | 0.005 | 0.005 | 0.005 |
| Flavor/sensate oils | 0.100 | 0.100 | 0.100 | 0.100 |
| Performathox 490 | 0.050 | 0.050 | 0.050 | 0.050 |
| Water | q.s. | q.s. | q.s. | q.s. |
| Total | 100% | 100% | 100% | 100% |
| Target pH | 6 | 6 | 6 | 6 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An oral care composition comprising:
a) from 0.01% to 5%, by weight of the composition, of a stannous ion source; and
b) from 0.01% to 10%, by weight of the composition, of an antifibrinolytic agent.

2. The oral care composition according to claim 1, wherein the antifibrinolytic agent is a compound of formula (I):
H₂N—X—COOH (I)
wherein X is a branched or unbranched, saturated or unsaturated, aliphatic group;
or an aromatic group.

3. The oral care composition according to claim 1, wherein the antifibrinolytic agent is present in an amount of less than 5%, by weight of the composition, and selected from tranexamic acid, epsilon aminocaproic acid, p-aminomethylbenzoic acid, or combinations thereof.

4. The oral care composition according to claim 3, wherein the antifibrinolytic agent is tranexamic acid, and present in an amount of from 0.1% to less than 1% by weight of the composition.

5. A oral care composition, comprising:
a) from 0.01% to 5%, by weight of the composition, of a stannous ion source; and
b) from 0.01% to 10%, by weight of the composition, of one or more compounds selected from the group consisting of tranexamic acid, epsilon aminocaproic acid, p-aminomethylbenzoic acid, and combinations thereof.

6. The oral care composition according claim 1, wherein the stannous ion source is present in the amount of from 0.05% to 4% by weight of the composition, and is selected from the group consisting of stannous chloride, stannous fluoride, stannous acetate, stannous gluconate, stannous oxalate, stannous sulfate, stannous lactate, stannous tartrate, stannous iodide, stannous chlorofluoride, stannous hexafluorozirconate, stannous citrate, stannous malate, stannous glycinate, stannous carbonate, stannous phosphate, stannous pyrophosphate, stannous metaphosphate, and combinations thereof.

7. The oral care composition according to claim 1, comprising from 0.01% to 5%, by weight of the composition, of a thickening agent comprising at least one agent selected from the group consisting of:
(i) a linear sulfated polysaccharide;
(ii) a natural gum;
(iii) a non-ionic cellulose or derivative thereof;
(iv) a polyvinyl pyrrolidone (PVP);
(v) a polymer comprising at least a polycarboxylated ethylene backbone;
(vi) polyacrylamide;
(vii) co-polymers comprising acrylamide;
(viii) pectin;
(ix) proteins;
(x) polyethylene glycols (PEG), preferably high molecular weight PEG; and
(xi) combinations thereof.

8. The oral care composition according to claim 7, wherein the thickening agent is present in an amount of from 1% to 2.5%, by weight of the composition, wherein:
(i) the linear sulfated polysaccharide is a carrageenan;
(ii) the natural gum is selected from the group consisting of xanthan gum, gum karaya, gum arabic, gum tragacanth, and combinations thereof;
(iii) the non-ionic cellulose or derivative thereof having an average molecular weight range of 50,000 to 1,300,000 Daltons, and preferably an average degree of polymerization from 300 to 4,800;
(iv) the polymer comprising at least a polycarboxylated ethylene backbone and selected from the group consisting of: co-polymers of maleic anhydride with methyl vinyl ether having a molecular weight of 30,000 to 1,000,000 Daltons; homo-polymers of acrylic acid; and co-polymers of maleic acid and acrylic acid or methacrylic; and
(v) combinations thereof.

9. The oral care composition according to claim 8, wherein:
(i) the carrageenan is selected from the group consisting of Kappa-carrageenan, Iota-carrageenan, Lambda-carrageenan, and combinations thereof;
(ii) the natural gum is xanthan gum;
(iii) the non-ionic cellulose or derivative thereof is hydroxyethyl cellulose (HEC);
(iv) the polymer comprising at least a polycarboxylated ethylene backbone is the co-polymers of maleic acid or anhydride with methyl vinyl ether; and
(v) combinations thereof.

10. The oral care composition according to claim 1, comprising from 0% to less than 0.001%, by weight of the composition folic acid.

11. The oral care composition according to claim 1 comprising: from 0.01% to 5% by weight of the composition, of a flavor composition, wherein the flavor composition comprises:
(i) a flavor mixture comprising from greater than 0% to less than 55%, or from greater than 65% to 95%, by weight of the flavor composition, of methyl salicyclate; from greater than 0% to less than 30%, or from greater than 35% to 65%, by weight of the flavor composition, of menthol; from greater than 0% to less than 1%, or from greater than 5% to 50%, by weight of the flavor composition, of eugenol; and from greater than 0% to less than 3%, or from greater than 8% to 30%, by weight of the flavor composition, of cineol; or
(ii) a flavor mixture that is free or substantially free of methyl salicyclate, menthol, eugenol, and cineol.

12. The oral care composition according claim 1 comprising from 0.0025% to 2% by weight of the composition, of a fluoride ion source, wherein the fluoride ion source is selected from the group consisting of sodium fluoride, indium fluoride, amine fluoride, sodium monofluorophosphate (MFP), potassium fluoride, zinc fluoride, and combinations thereof.

13. The oral care composition according to claim 1 comprising from 10% to 70% water.

14. The oral care composition according to claim 1, wherein the composition has a pH from 4.5 to 11.

15. A method of promoting Gum Health in a human subject comprising administering to the subject's oral cavity an oral care composition according to any one of claims 1 to 14, preferably once a day, more preferably twice a day.

16. The method of claim 15, wherein the promotion of Gum Health occurs within a period selected from the group consisting of:
a) from time 0 hours to 72 hours;
b) from time 0 hours to 48 hours;
c) from time 0 hours to 24 hours;
wherein time 0 hours is upon the administration of the oral care composition.

17. The method of claim 15 or 16, wherein Gum Health is selected from:
(i) improving gingival wound healing in the oral cavity;
(ii) improving reduction of bacterial activity in the oral cavity; or
(iii) combination thereof.
